(19) United States
(12) Patent Application Publication
(10) Pub. No.: US 2026/0047853 A1
(43) Pub. Date: Feb. 19, 2026

(54) GEAR DRIVEN ANGLED AWL MEDICAL INSTRUMENT

(71) Applicant: Warsaw Orthopedic, Inc., Warsaw, IN (US)

(72) Inventors: Darren L. Davis, Millington, TN (US); Kendra M. Dawson, Memphis, TN (US)

(21) Appl. No.: 19/283,489

(22) Filed: Jul. 29, 2025

Related U.S. Application Data
(60) Provisional application No. 63/682,853, filed on Aug. 14, 2024.

(51) Int. Cl. A61B 17/16; A61B 17/00; A61F 2/46

(52) U.S. Cl. CPC A61B 17/1671; A61F 2/4611; A61B 2017/00367

(57) ABSTRACT

A surgical device having an angled awl is disclosed. The surgical device includes an outer shaft extending between a proximal end and a distal end along a central axis. An angled sleeve is positioned on the distal end and is angled relative to the central axis. The outer shaft houses a driving mechanism including an inner shaft and one or more driving elements configured to translate a bone piercing tool along a trajectory between a non-deployed position and a deployed position upon actuation of a push button. The bone piercing tool is housed within the angled sleeve so that a trajectory of the bone piercing tool forms an angle with the central axis. The angled sleeve also houses a first gear and a second gear, the second gear being coupled to the bone piercing tool and the first gear and second gear being oriented at an angle relative to each other.

### CROSS-REFERENCE TO RELATED APPLICATION

This application claims the benefit of and priority to U.S. Provisional Patent Application No. 63/682,853 filed Aug. 14, 2024, the entire disclosure of which is incorporated by reference herein.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, they may fail to relieve the symptoms associated with these disorders. Surgical treatment of these spinal disorders includes fusion, fixation, correction, discectomy, laminectomy and implantable prosthetics. As part of these surgical treatments, spinal constructs, such as, for example, bone fasteners, spinal rods and interbody devices can be used to provide stability to a treated region. For example, during surgical treatment, interbody devices may be introduced to a space between adjacent vertebral bodies (the interbody space) to properly space the vertebral bodies and provide a receptacle for bone growth promoting materials, e.g., grafting. The interbody devices may be fixed to one or both adjacent vertebrae and fastened to the vertebra, via bone fasteners inserted through a respective fastener hole of the interbody device.

To fasten an implant or medical device to the vertebra, a user may use a medical access instrument, such as an awl, to create a hole in the vertebra or other bony structure to insert a fastener or to otherwise secure the implant or medical device into or through a bone. Awls are especially useful for creating pilot holes. Furthermore, pilot holes are advantageous because they reduce the risk of bone cracking during screw or fastener insertion and minimize the amount of torque necessary for screw or fastener insertion. Pilot holes also help direct the screw or fastener along the intended trajectory. The result is improved implant and/or medical device stability and fewer complications. However, certain conventional awls are substantially linear. Thus, certain linear extension awls may not be able to place a pilot hole or fastener hole at the target location, as the target location may be obstructed by other bodily objects within the patient. Furthermore, linear awls are not always capable of creating angled pilot holes or fastener holes. Thus, an advancement of angled awls is that they are capable of not only of crafting angled fastener holes but also enhancing case of access in difficult-to-reach areas within bony structures having limited clearance to perform the installation. Another advantage of the present disclosure is a hyper efficient transmission of power through the awl to create a pilot hole.

The present disclosure seeks to address these and other shortcomings in the existing relevant arts.

### SUMMARY

The Techniques of this Disclosure Generally Relate to

In one aspect, the present disclosure includes a surgical device, having an outer shaft extending between a proximal end and a distal end along a central axis; a driving mechanism including an inner shaft having a first threaded portion and extending through the outer shaft along the central axis between the proximal end and the distal end; and one or more driving elements in contact with the first threaded portion of the inner shaft. The surgical device also includes an angled sleeve positioned on the distal end of the outer shaft and supporting an angled shaft therein that is angled relative to the central axis; a first gear coupled to a distal end of the inner shaft and a second geared meshed with the first gear, the second gear coupled to the angled shaft; a push button coupled to the proximal end of the inner shaft, wherein the push button supports the one or more driving elements and is linearly translatable between a first position and a second position; and a bone piercing tool coupled to the angled shaft and rotatable via rotation of the second gear, the bone piercing tool being extendable and retractable along a trajectory between a non-deployed position and a deployed position upon linear translation of the push button, in the deployed position, a portion of the bone piercing tool extends distally beyond a distal tip of the angled sleeve along the trajectory. When linearly translating from the first position to the second position, the push button is configured to simultaneously move the one or more driving elements in a forward direction along the first threaded portion so that the inner shaft axially rotates in a first direction and linearly translates the bone piercing tool along the trajectory to the deployed position. When linearly translating from the second position to the first position, the inner shaft axially rotates in a second direction opposite the first direction and linearly translates the bone piercing tool along the trajectory to the non-deployed position.

In another aspect, the present disclosure includes a surgical device including a rotational mechanism configured to translate rotational force and a bone piercing tool coupled to the rotational mechanism and configured to rotate upon translation of rotational force to the bone piercing tool by the rotational mechanism. The bone piercing tool includes a threaded portion and one or more posts mated with the threaded portion, the one or more posts being configured to cause the bone piercing tool to linearly move back and forth between a non-deployed position and a deployed position when the rotational mechanism translates rotational force to the bone piercing tool.

In another aspect, the present disclosure includes a medical device system, including a medical implant comprising at least one fastening hole and a surgical device. The surgical device includes an outer shaft extending between a proximal end and a distal end along a central axis; a driving mechanism having an inner shaft having a first threaded portion and extending through the outer shaft along the central axis between the proximal end and the distal end; and one or more driving elements in contact with the first threaded portion of the inner shaft. An angled sleeve is positioned on the distal end of the outer shaft and supports an angled shaft therein that is angled relative to the central axis. A first gear is coupled to a distal end of the inner shaft and a second geared is meshed with the first gear, the second gear being coupled to the angled shaft. A push button is coupled to the proximal end of the inner shaft, wherein the push button supports the one or more driving elements and is linearly translatable between a first position and a second position. A bone piercing tool is coupled to the angled shaft and is rotatable via rotation of the second gear, the bone piercing tool being extendable and retractable along a trajectory between a non-deployed position and a deployed position upon linear translation of the push button. In the deployed position, a portion of the bone piercing tool extends distally beyond a distal tip of the angled sleeve along the trajectory. When linearly translating from the first position to the second position, the push button is configured to simultaneously move the one or more driving elements in a forward direction along the first threaded portion so that the inner shaft axially rotates in a first direction and linearly translates the bone piercing tool along the trajectory to the deployed position. When linearly translating from the second position to the first position, the inner shaft axially rotates in a second direction opposite the first direction and linearly translates the bone piercing tool along the trajectory to the non-deployed position. The bone piercing tool is configured to cut holes in bony anatomy corresponding to the size and angularity of the at least one fastening hole.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The following drawings are illustrative of particular embodiments of the present disclosure and therefore do not limit the scope of the present disclosure. The drawings are not to scale and are intended for use in conjunction with the explanations in the following detailed description.

FIG. 1A illustrates a perspective view of an example surgical device;

FIG. 1B illustrates an alternative perspective view of an example surgical device in an extended position;

FIG. 2 illustrates an exploded view of an example surgical device;

FIG. 3A is a cross-sectional view of an example surgical device taken through plane A-A of FIG. 1B. The surgical device includes a push button in an actuated position and a bone piercing tool in a deployed position.

FIG. 3B is a cross-sectional view of an example surgical device taken through plane A-A of FIG. 1B. The surgical device includes a push button in an unactuated position and a bone piercing tool in a non-deployed position.

FIG. 3C is an enlarged view of a portion of the example surgical device shown in FIGS. 3A-B.

FIG. 4 illustrates an example of a push button for use with a surgical device;

FIG. 5 illustrates an example of a cap for use with a surgical device;

FIG. 6A illustrates a distal end of an example surgical device with some parts removed for ease of understanding;

FIG. 6B is an alternative view of the distal end of FIG. 6A where axes are shown to illustrate the angle of orientation between various components of the example surgical device;

FIG. 7A is a cross-sectional view through a portion of an example surgical device. A bone piercing tool is shown in the deployed position.

FIG. 7B is a cross-sectional view through a portion of an example surgical device. A bone piercing tool is shown in the non-deployed position.

FIG. 8 illustrates a cross-sectional view taken through the natural center line of an angled sleeve for use with a surgical device;

FIG. 9 illustrates the distal end of an example surgical device with some parts removed for ease of understanding;

FIG. 10A illustrates an example of an outer shaft for use with a surgical device having cross section line IA through the natural center line of the outer shaft;

FIG. 10B illustrates a cross-sectional view taken through cross section line IA of FIG. 10A;

FIG. 11 illustrates an example of an inner shaft for use with a surgical device;

FIG. 12A illustrates an example of a thrust bearing for use with a surgical device;

FIG. 12B illustrates an example of a distal bushing for use with a surgical device; and

FIG. 13 illustrates an example of an inner shaft and a push button for use with a surgical device.

### DETAILED DESCRIPTION

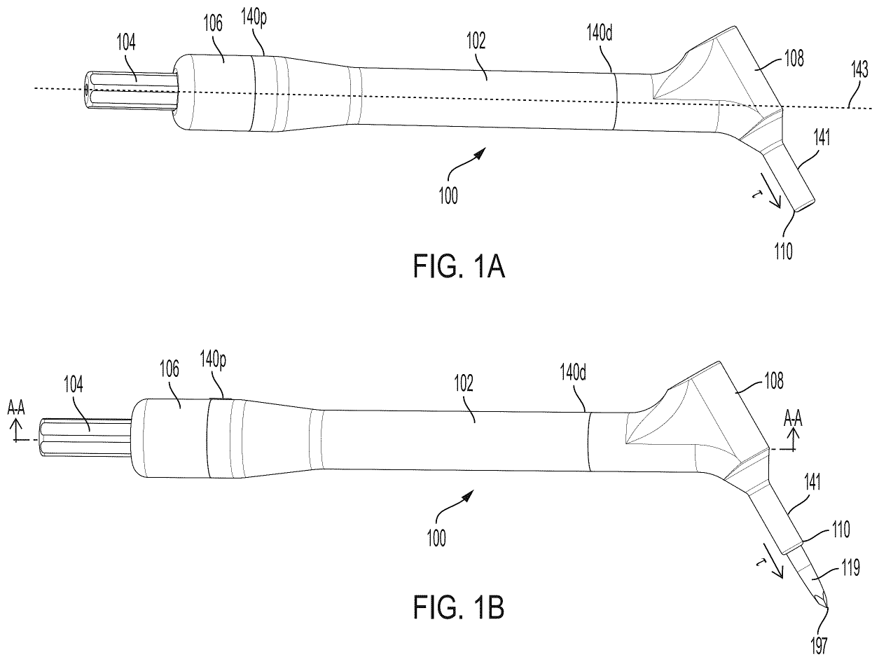

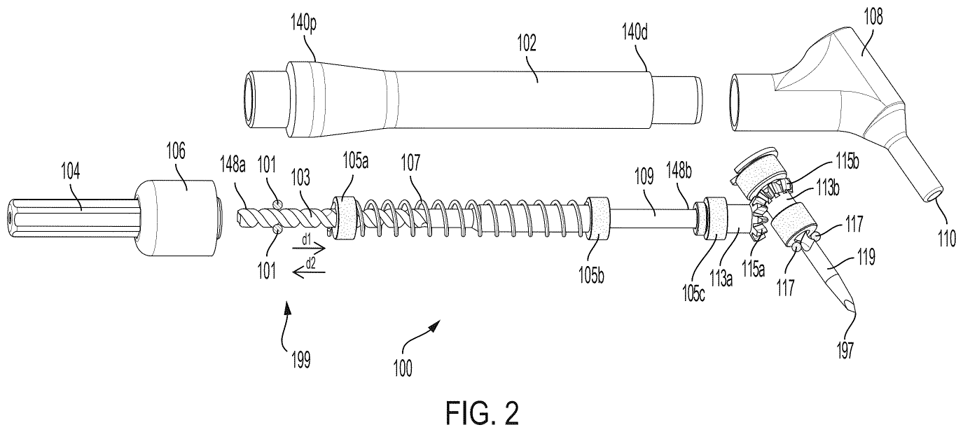

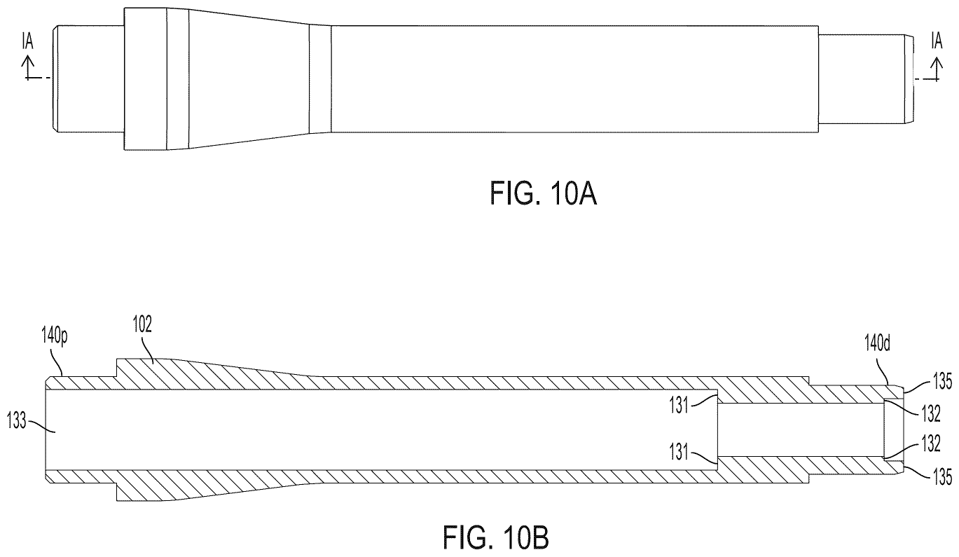

FIGS. 1A and 1B show an embodiment of a surgical device 100. The surgical device 100 includes an outer shaft 102 extending between a proximal end 140p and a distal end 140d along a central axis 143. The outer shaft 102 includes a hollow interior 133 (shown in FIG. 10A-B) for housing surgical device components, discussed in more detail in relation to FIG. 2 and later figures. At the proximal end of the surgical device 100 is a push button 104 at least partially enclosed by a cap 106. In the embodiment shown in FIGS. 1A and 1B the cap 106 radially surrounds a portion of the push button 104 while another portion of the push button 104 extends beyond the cap 106 away from the proximal end 140p. In this manner the cap 106 serves the dual-purpose of forming a protective outer sheath for the push button and couples the push button to the outer shaft 102. Proceeding along the outer shaft 102 to the distal end 140d is an angled sleeve 108 having a cylindrical portion 141. The cylindrical portion 141 also includes a hollow interior 133. The cap 106, outer shaft 102, and angled sleeve 108 may be manufactured as a single cohesive component (i.e., unitary component) or as multiple components coupled together e.g., by threading.

A bone piercing tool 119 is housed within the angled sleeve so that the bone piercing tool 119 extends through the hollow interior 133 of the cylindrical portion 141. The bone piercing tool 119 includes a tip 197 which may include a trocar tip, drill tip, driver tip, burr tip, awl tip, reamer tip or other configuration suitable to cut through bone or puncture through bone. The bone piercing tool may be configured to cut holes in a substrate. Such holes may extend at an angle through the substrate allowing the bone piercing tool 119 to cut holes and/or punch holes that align with an angled aperture on another surgical component such as an inter-body device.

The bone piercing tool 119 may be removed from surgical device 100 and interchanged with another bone piercing tool. Therefore, the user has the flexibility to select the appropriate tip (bone piercing tool 119) according to the specific procedure, bone density, required hole diameter, or user preference. Moreover, bone piercing tool 119 may incorporate materials such as titanium alloys or diamond-coated surfaces to enhance durability, cutting efficiency, and biocompatibility. Some bone piercing tool 119 designs may also feature integrated irrigation channels or depth stop mechanisms to further improve safety and precision during bone penetration.

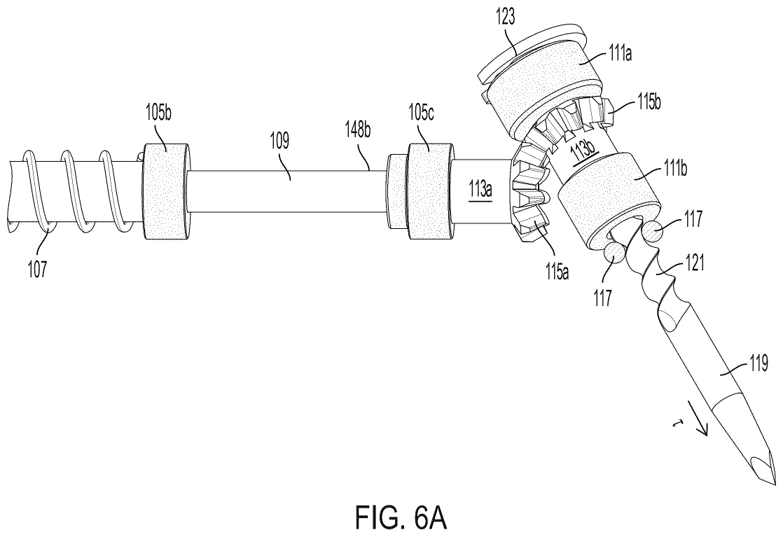

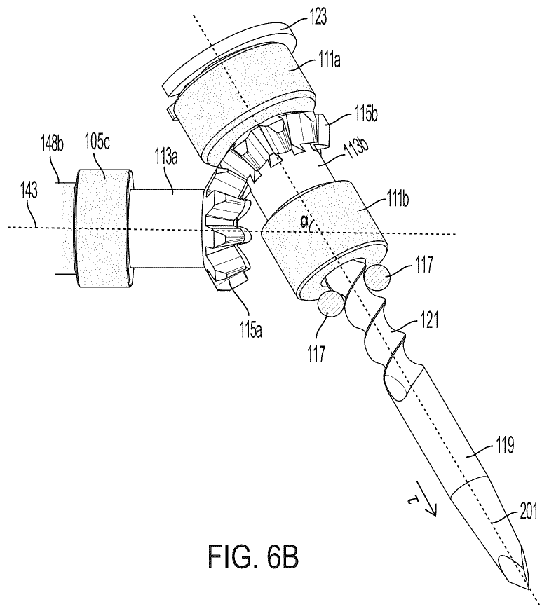

Upon actuation of the push button 104, the bone piercing tool 119 transitions from a non-deployed position (FIG. 1A) to a deployed position (FIG. 1B). Specifically, the bone piercing tool 119 transitions from its non-deployed or retracted position within the angled sleeve 108 to an operational state in the deployed position where it extends beyond the distal tip 110 of the cylindrical portion 141 of the angled sleeve 108. Conversely, upon deactivation or release of the push button 104, the bone piercing tool 119 reverts from the operational deployed position back to the retracted non-deployed position. More detail on the mechanisms governing movement of the bone piercing tool 119 are discussed in more detail in relation to FIGS. 2, 6A-B and 7A-B.

FIG. 1A shows the bone piercing 119 tool in a non-deployed position i.e., the tip is retracted so that it is concealed by the angled sleeve 108 and does not extend beyond the distal tip 110 of the angled sleeve. FIG. 1A shows the bone piercing tool in the non-deployed position. The non-deployed position allows the bone piercing tool 119 to be stored safely and securely within the surgical device 100 and to not cause interference e.g., unwanted cutting during transport, storage or a surgical procedure when not in use. FIG. 1B shows the bone piercing tool 119 in the deployed position e.g., extending beyond the distal tip 110 of the angled sleeve 108. The deployed position allows the bone piercing tool 119 to extend beyond the distal tip 110 of the angled sleeve sufficiently enough to cut or pierce through a substrate.

FIG. 2 shows an exploded view of the surgical device 100. We will now discuss the components housed within outer shaft 102. Housed within outer shaft 102 is inner shaft 109. Inner shaft 109 is coaxially aligned with outer shaft 102. Inner shaft 109 extends between proximal end 140p to distal end 140d inside of the hollow outer shaft 102. Inner shaft includes a first end 148a located near the proximal end 140p of the outer shaft 102 and a second end 148b located near the distal end 140d of outer shaft 102. Near or adjacent to first end 148a there is a threaded portion 103 on the inner shaft 109. The threaded portion 103 includes threads configured to interact with driving elements 101. Apart from the threaded portion 121, the surface of inner shaft 109 is smooth or relatively smooth i.e., lacking threads. A spring 107 e.g., an clastic spring, partially encloses inner shaft 109. In the illustrated embodiment the spring 107 is near the first end 148a and partially encloses threaded portion 103. Thrust bearings 105a-c, three in the illustrated embodiment (but more or fewer are contemplated), radially surround inner shaft 109 and are configured to prevent undesired translation of the inner shaft 109 within outer shaft 102.

Thrust bearing 105a surrounds inner shaft 109 at or proximate the threaded portion 103. Thrust bearing 105a may translate with push button 104 when push button 104 is actuated. Thrust bearing 105b surrounds inner shaft 109 downstream (towards second end 148b) of thrust bearing 105a and serves as a fixed bearing surface for spring 107 when it is compressed by push button 104 i.e., spring 107 is compressed between thrust bearing 105a and thrust bearing 105b when push button 104 is actuated. Thrust bearing 105c surrounds inner shaft 109 further downstream (towards second end 148b) of thrust bearing 105b. In the illustrated embodiment, thrust bearing 105c is partially housed within outer shaft 102 and angled sleeve 108. Thrust bearing 105c couples shaft 113a of gear 115a (discussed in detail later) to inner shaft 109. The interaction between the thrust bearings 105a-c, outer shaft 102, and the inner shaft 109 is discussed in more detail in relation to FIGS. 10A-B and 11. Furthermore, a person of skill in the art would recognize that many different types of bearings can be used with surgical device 100.

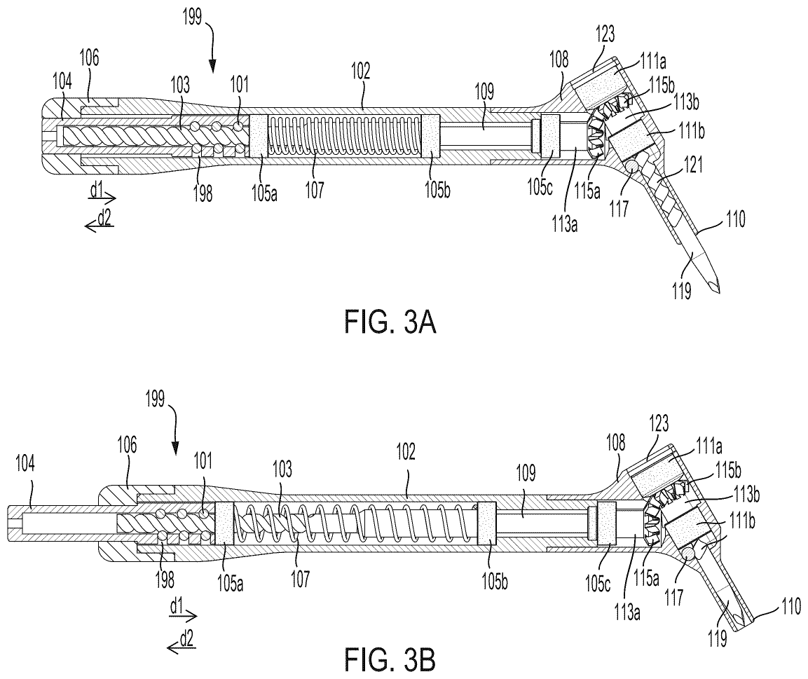

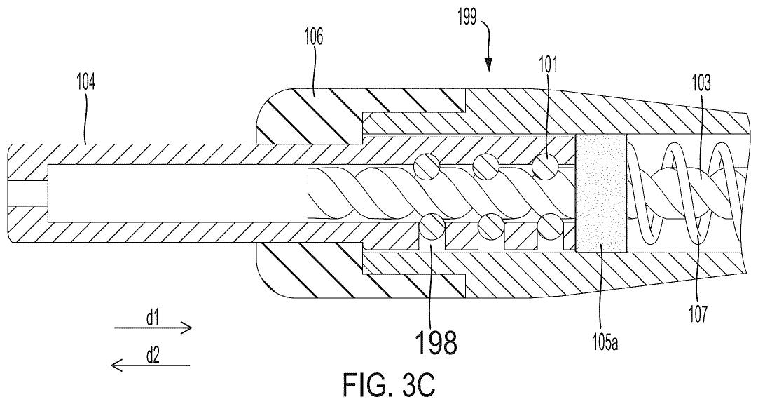

In the embodiment illustrated in FIG. 2, two driving elements 101 are shown. However, other embodiments may include more or fewer driving elements e.g., 1, 2, 3, 4, 5, 6, 7, 8, 9 driving elements. Varying the number of driving elements 101 allows for variation of the amount of rotational translation of the inner shaft 109 per specific amount of linear actuation of the push button 104. The driving elements 101 may each be housed in respective corresponding pockets, indents, slots, or grooves 198 (see FIGS. 3A-C) in the interior of push button 104; and the corresponding pocket, indent, or groove 198 allows the driving elements 101 to contact the threaded portion 103 and to freely rotate in place therein without translating linearly relative to the push button 104. With the driving elements being capable of spinning in place in the corresponding pocket (and remaining in place linearly relative to the push button 104) when the push button 104 is translated forward linearly a resultant rotational force is applied to the inner shaft 109. In the illustrated embodiment the driving elements are balls that are meshed with threads on the threaded portion 103. In other embodiments the driving elements 101 may be teeth, such as those that are typical of acme screws, for example, and the teeth may track with the threads of the threaded portion 103. Collectively, the driving elements 101, threaded portion 103, and inner shaft 109 form a driving mechanism 199 that can actuate the gears 115b in the angled sleeve 108. In the example embodiment, the driving elements 101 interact with the threaded portion 103 so as to cause the inner shaft 109 to rotate and the rotation of the inner shaft 109 causes the bone piercing tool 119 to simultaneously rotate and linearly translate forward (extend beyond the angled sleeve 108). The specific interaction between the driving elements 101, the inner shaft 109, and the bone piercing tool 119 is discussed in more detail in the forthcoming paragraphs.

Translating the bone piercing tool 119 by means of the push button 104 eliminates the need to use a hammer to drive the bone piercing tool 119 into a substrate, as is common with traditional awls. This arrangement reduces the footprint required to operate the surgical device 100 and minimizes risk related to hammering during surgical procedures. Furthermore, the push button 104 may be manually actuated by an end user during operation of surgical device 100. Additionally or alternatively, the push button 104 may be actuated automatically by means of a pneumatic cylinder, a hydraulic punch or an electronic or powered device. Still further, the push button 104 may have a t-handle attached to it so that it may be actuated by pushing on the t-handle or the bone piercing tool 119 may be translated by actuation of a t-handle in place of the push button 104.

Downstream of the threaded portion 103 and the spring 107, proximal the second end 148b of inner shaft 109, are a pair of gears 115a-b each having a respective gear shaft 113a-b of which they are fixed to. Gear shafts 113a and 113b may be cannulated, the cannula being configured to receive other components of surgical device 100. The gears 115a-b may be bevel gears or miter gears, for example. In some embodiments, each shaft 113a-b is positioned at an angular separation of about 50-70 degrees from another or about 60 degrees from one another. In other embodiments the shafts 113a-b may be positioned at nearly exactly a 60-degree angle while at the same time the gears 115a-b consist of teeth typical to those of miter gears i.e., the same number of teeth on each gear 115a-b forming a 1:1 gear ratio. As the shafts 113a-b rotate during operation of the surgical device 100 the teeth of each of the respective gears 115a-b also rotate. The teeth of each of the gears 115a-b are meshed so that as one of the gears 115a-b rotates its counterpart rotates. The angular configuration of the gears 115a-b facilitates mechanical efficiency and optimizes space within the constrained space of the angled sleeve 108. The angular configuration also allows the surgical device 100 to cut angled holes into bone or another substrate. This allows the user to create pilot holes using surgical device 100 that accommodate angled fastener holes on implants and other inter-body devices. In addition, the angular configuration allows a user, such as a surgeon, to access difficult to reach portions of a substrate during surgical procedures i.e., portions of substrate that are far away from the incision or obstructed by anatomy or other surgical tools.

In some embodiments the gears 113a-b may be substituted for a different rotational mechanism. Such mechanism may include a worm and gear, a flex shaft or a one or more ball joints. These rotational mechanisms may interact with one or more shafts the same as similar to or substantially similar to shafts 115a-b to transfer rotational force so as to effectuate rotation and/or linear movement of the bone piercing tool 119, which may be coupled to one or more components of the rotational mechanism. The bone piercing tool may be oriented at angle, a, relative to central axis 143 in embodiments including these rotational mechanisms.

It should also be noted that the gear ratio may be adjusted to increase or decrease the speed at which the bone piercing tool 119 rotates relative to upstream components. For example, the gear ratio of gear 115a to gear 115b may be 3:1 so that gear 115b and bone piercing tool 119 rotate slower than gear 115a and inner shaft 109. Conversely, the gear ratio of gear 115a to gear 115b may be 1:3 so that gear 115b and bone piercing tool 119 rotate faster than gear 115a and inner shaft 109.

As can be seen in FIG. 2, one of the gears 115a is coaxially aligned with the inner shaft 109. Gear 115a is fixedly coupled directly to inner shaft 109 and therefore rotates an equal amount. A second gear 115b is located downstream of gear 115a and is coupled to bone piercing tool 119 (as opposed to inner shaft 109). Second gear 115b is oriented at an angle relative to inner shaft 109 and first gear 115a i.e., its shaft 113b is oriented at an angle α (see FIG. 6B) relative to shaft 113a and inner shaft 109. The teeth of gear 115a intersect with the valleys of gear 115b so that as gear 115a rotates, gear 115b also rotates. Because it is coupled to inner shaft 109, first gear 115a rotates when inner shaft 109 rotates. Furthermore, because the bone piercing tool 119 is coupled to the second gear 115b, the intersection between gears 115a and 115b subsequently causes the bone piercing tool to rotate.

When the push button 104 is actuated the driving elements 101 are forced to rotate in their respective pockets, idents, or grooves 198 (shown in FIGS. 3A-C) within the push button 104. Because they are in contact with threaded portion 103, as the driving elements 101 rotate during push button actuation they track with the threads of the threaded portion 103 and cause the inner shaft 109 to rotate in a first direction (e.g., clockwise or anti-clockwise). While the push button 104 is actuated and the driving elements 101 are forced to rotate the spring 107 is compressed by the push button 104. The spring 107 may compress, at least in part, because it is flanked between the thrust bearings 105a and 105b and/or the push button 104. Thrust bearing 105a translates with the push button 104 and thrust bearing 105b is stationary providing the spring 107 a fixed surface against which to compress. Thrust bearing 105b may be stationary by virtue of being disposed within a pocket, indent, or groove within the interior of shaft 102. Conversely, when the push button 104 is released the compressed spring 107 forces the push button 104 and/or thrust bearing 105a upstream the inner shaft 109 towards first end 148a and the driving elements 101 rotate in the opposite direction compared to when the push button 104 was actuated causing the inner shaft to rotate in a second direction. In some embodiments, the inner shaft may rotate in a clockwise direction when looking down the central axis 143 from the proximal end 140p when the push button 104 is actuated and in an anti-clockwise direction when the push button 104 is released and vice versa.

When the inner shaft 109 rotates upon actuation and release of the push button 104 the upstream reaction involving the spring 107, driving elements 101, and inner shaft 109 causes the first and second gears 115a and 115b to rotate thereby causing the bone piercing tool 119 to transition between the non-deployed position (when the push button 104 is actuated) and the deployed position (when the push button 104 is released). The interaction of these components causes the bone piercing tool 119 to rotate as it linearly translates (extends beyond the distal tip 110).

The interaction between the push button 104, the spring 107 and the driving mechanism 199 (the driving elements 101, the inner shaft 109, and the threaded portion 103) results in back driving behavior of the driving mechanism 199 when the push button 104 is compressed and released i.e., the linear motion of the push button 104 is translated to rotational motion of the inner shaft 109. This back driving behavior makes it easier to translate the bone piercing tool 119 from the non-deployed position to the deployed position while the bone piercing tool is oriented at an angle relative to the central axis 143 (see FIG. 1B).

FIG. 3A is a cross-sectional view of surgical device 100 taken through plane A-A of FIG. 1B. Bone piercing tool 119 is in the deployed position and extends beyond distal tip 110. The interaction of push button 104 with driving mechanism 199 and spring 107 is shown. Push button 104 is at least partially housed within recess 114 of outer shaft 102 (shown in FIG. 5) and can move linearly within outer shaft 102 along central axis 143. Push button 104 is actuated in direction d1 i.e., push button is actuated into recess 114 of cap 106. Push button 104 exerts force on thrust bearing 105a so as to compress spring 107. Driving elements 101 move along the threads of threaded portion 103 in direction d1 so that they are closer to second end 148b of inner shaft 109 relative to when push button 104 is unactuated. Driving elements 101 rotate in their respective pockets 198 in the body of push button 104 so as to move along threaded portion 103. Note driving elements 101 are in contact with threaded portion 103 while they are received in their respective pockets 198. Pockets 198 may be configured as indents, grooves, windows or holes of push button 104 and may be configured to hold driving elements 101 securely within push button 101 while providing sufficient space for driving elements 101 to rotate or roll in directions d1 and d2 along threaded portion 103 while remaining translationally stationary relative to push button 104.

FIG. 3B is a cross-sectional view of surgical device 100 taken through plane A-A of FIG. 1B. Bone piercing tool 119 is in the non-deployed position i.e., is concealed or substantially concealed in angled sleeve 108. Push button 107 is unactuated and extends beyond cap 106. Spring 107 is in a relaxed or uncompressed state. Driving elements 101 move along the threads of threaded portion 103 in direction d2 (opposite direction d1) when force is removed from push button 104 and spring 107 decompresses. Spring 107 biases thrust bearing 105a and push button 104 to the positions shown in FIG. 3A. Driving elements 101 move along the threads of threaded portion 103 in direction d2 so that they are closer to first end 148a of inner shaft 109 relative to when push button 104 is unactuated. Driving elements 101 rotate in their respective pockets 198 in the body of push button 104 so as to move along threaded portion 103.

FIG. 3C is an enlarged view of the push button of FIG. 3B.

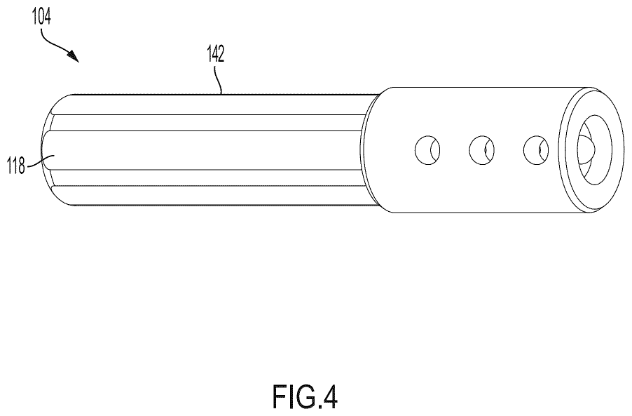

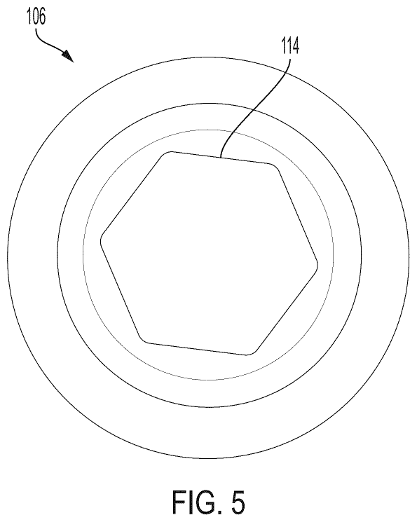

Referring now to FIGS. 4 and 5 push button 104 and cap 106 are respectively shown. As shown in FIG. 4, push button 104 includes a body 142 having a plurality of planar or substantially planar sides 118 disposed around the body 142. As best shown in FIG. 5, cap 106 (with the push button 104 removed) includes hexagonal recess 114 in which push button 104 is received. The hexagonal recess 114 meshes with the sides 118 of push button 104 when push button 104 is received in the recess 114 of cap 106. This meshing prevents push button 104 from rotating within cap 106 thereby preventing the driving elements 101 from inadvertently causing inner shaft 109 to rotate.

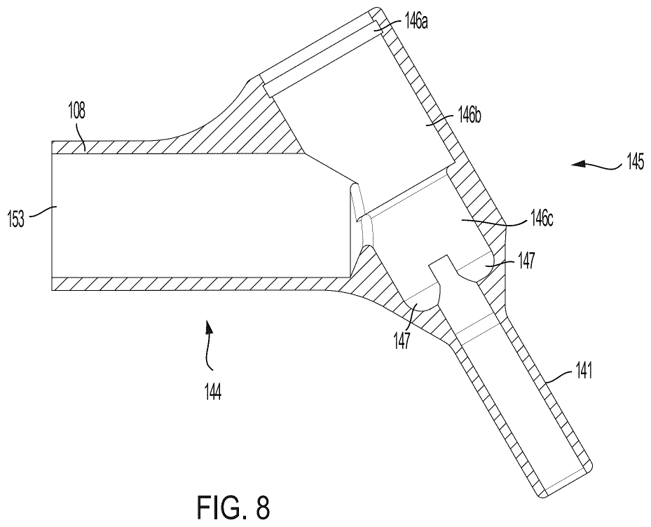

There are additional mechanisms that govern the means by which the bone piercing tool 119 moves between the non-deployed and deployed positions which will now be discussed in relation to FIGS. 6A-B, 7, 8 and 9. FIG. 6A, shows a zoomed in view of the distal end 140d of surgical device 100. Parts have been removed for ease of understanding. As can be seen in FIG. 6A one or more balls 117 (or posts) are shown and are in contact with a threaded portion 121 of bone piercing tool 119. The balls 117 are each housed in a corresponding pocket 147 (shown in FIG. 8) of the angled sleeve 108 and are flanked between the bottom of the corresponding pockets 147 and the distal bushing 111b. A thrust bearing 105c couples the shaft 113a of gear 115a to second end 148b of the inner shaft 109. Distal bushings 111a and 111b flank gear 115b. Thrust bearing 105c prevents gear 115a from translating within angled sleeve 108. Distal bushings 111a and 111b ensure proper alignment of gear 115a and 115b relative to each other and ensure proper translation of the bone piercing tool 119 between the non-deployed and deployed positions. Specifically, distal bushing 111a prevents translation of gear 115b away from the distal tip 110 distal busing 111b prevents translation of gear 115b towards distal tip 110. There is a retaining clip 123 adjacent distal bushing 111a to secure it in place within angled sleeve 108 as a backstop.

Referring now to FIG. 6B, an alternative zoomed in view of the distal end 140d of surgical device 100 is shown. Once again, parts have been removed for case of understanding. Shaft 113a is oriented (on inner shaft 109) along central axis 143. Shaft 113b is oriented (within angled sleeve 108) along axis 201, which may coincide with trajectory t of bone piercing tool 119. For example, the rotation axis of shaft 113b and bone piercing tool 119 are coaxially aligned. Bone piercing tool 119 may rotate about axis 201 as it linearly translates between the deployed and non-deployed positions. Central axis 143 (of shaft 109) and axis 201 form an angle α relative to one another. Accordingly, shaft 113a and shaft 113b are also oriented to one another at angle α. Stated another way, central axis 143 and bone piercing tool 119 (oriented along trajectory t) are oriented relative to one another at angle α. Angle α may fall within a range of about 15 degrees to about 90 degrees. An advantage of the back driving behavior of the driving mechanism 199 is that it allows the bone piercing tool 119 to translate between the deployed and non-deployed positions at a higher angle α (about 60 degrees or greater). Furthermore, a higher angle α may be advantageous because it better allows a surgeon or end user of the surgical device 100 to install screws or other fasteners into a surgical site that is farther away, obstructed, or otherwise difficult to access.

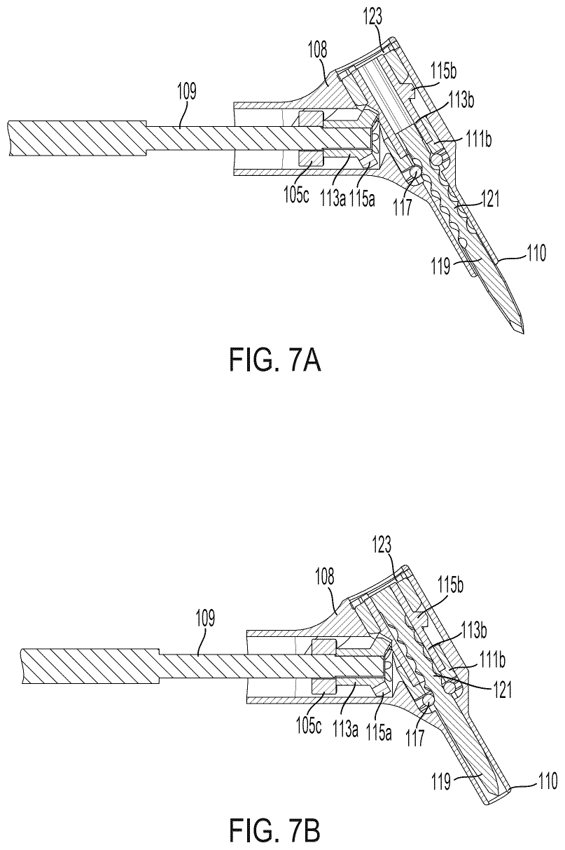

FIG. 7A is a cross-sectional view of angled sleeve 108 taken through the natural centerline of angled sleeve 108. Bone piercing tool 119 is shown in the deployed position. Inner shaft 109 is at least partially received in a hollow interior of shaft 113a. Bone piercing tool 119 is at least partially received in a hollow interior of shaft 113b. As described previously herein, as inner shaft 109 rotates, gear 115a and shaft 113a rotate. Gear 115a is meshed with gear 115b so that gear 115b and shaft 113b also rotate as inner shaft 109 rotates. Bone piercing tool 119 and shaft 113b each have a corresponding hexagonal shape allowing bone piercing tool to remain stationary relative to shaft 113b as shaft 113b rotates. As bone piercing tool 119 rotates with shaft 113b balls 117 rotate in pockets 147 and simultaneously contact threaded portion 121 allowing bone piercing tool 119 to extend linearly along trajectory t as it rotates with shaft 113b. When bone piercing tool 119 moves from the non-deployed position to the deployed position balls 117 move up threaded portion 121 away from tip 197, or stated another way, in a direction opposite the direction of extension of bone piercing tool 119 along trajectory t.

FIG. 7B is a cross-sectional view of angled sleeve 108 taken through the natural centerline of angled sleeve 108. Bone piercing tool 119 is shown in the non-deployed position. When bone piercing tool 119 moves from the deployed position to the non-deployed position balls 117 move down threaded portion 121 towards tip 197, or stated another way, in a direction opposite the direction of retraction of bone piercing tool 119 along trajectory t.

Referring now to FIG. 8, a cross section of angled sleeve 108 is shown. The cross section is taken through the natural centerline of angled sleeve 108. Interior components of outer shaft 102 have been removed for ease of understanding. Angled sleeve 108 includes a linear portion 144 having a recess 153 for housing gear 115a, thrust bearing 105c, and neck portion 154 of outer shaft 102. Linear portion 144 extends in a direction parallel to central axis 143 from distal end 140d of outer shaft 102. Extending at an angle relative to linear portion 144 is angled portion 145. Angled portion 145 culminates in a tip of the device, i.e., cylindrical portion 141. Angled portion 145 houses several components including gear 115b, distal bushings 111a and 111b, bone piercing tool 119 and balls 117 (see also FIG. 6B). Angled portion 145 includes groove 146a for stabilizing retaining clip 123 in place. Angled portion 145 also includes grooves 146b and 146c for stabilizing shaft 113b and distal bushing 111b in place, respectively. Furthermore, angled portion 145 includes pockets 147 that each house a corresponding ball 117 that may freely spin in place in the corresponding pocket 147. In particular, each ball 117 is prevented from moving out of its corresponding pocket 147 because it is trapped within its pocket 147 by distal bushing 111b. In the embodiment illustrated in FIG. 8, the pockets 147 have a curved profile to closely fit the curved profile of the balls 117. As discussed previously, the number of pockets 147 can vary with the number of balls 117. The angled sleeve 108 in FIG. 8 shows two pockets 147 which each house a single ball 117, but other embodiments may include three pockets 147 each housing one of three balls 117 if so desired. Bone piercing tool 119 is housed in angled portion 145 and extends along the length of angled portion 145 through distal bushings 111a and 111b and shaft 113b through cylindrical portion 141.

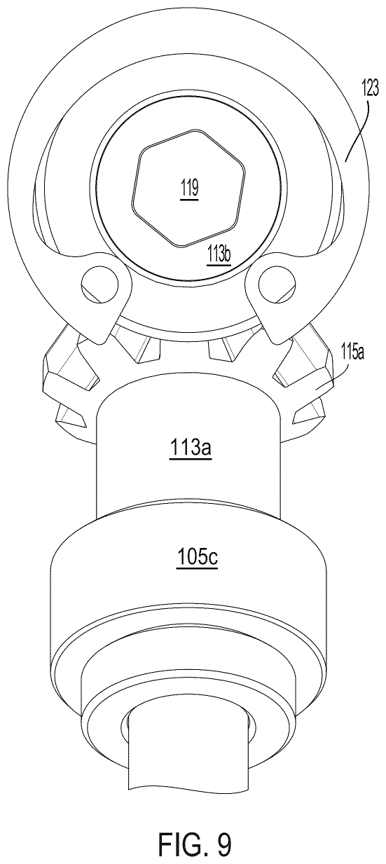

As best shown in FIG. 9, the bone piercing tool 119 has a hexagonal cross-section that is keyed with the interior of shaft 113b so that the bone piercing tool 119 rotates with the shaft 113b yet does not rotate relative to shaft 113b. The bone piercing tool being keyed to the interior of the shaft 113b allows the bone piercing tool 119 to extend and retract along a trajectory, t, within the angled sleeve 108 when the gear 115b rotates and the balls 117 interact with the threaded portion 121.

In the illustrated embodiment, two balls 117 are shown, however more or less balls each having a corresponding pocket are contemplated. For example, in some embodiments the threaded portion 121 may be a dual-lead or multi-lead thread. In such embodiments there may be one ball 117 corresponding to each thread i.e., a dual-lead thread having two balls 117 or a triple lead thread having three balls 117. A dual-lead thread may be advantageous because pressure is exerted evenly on each of the two balls 117 by the threaded portion 121. A dual-lead thread or multi-lead thread may be advantageous because it allows the bone piercing tool 119 to extend a greater distance beyond the distal tip 110 per revolution of the gears 115a and 115b.

Referring now to FIG. 10A-B, a cross section through cross section line IA or the natural center line of outer shaft 102 is shown. Interior components of outer shaft 102 have been removed for ease of understanding. Outer shaft 102 includes hollow interior 133 which houses the inner shaft 109, push button 104, gears 115a and 115b, and spring 107. The outer shaft 102 also includes shoulders 131 against which thrust bearing 105b rests. Shoulders 131 are configured of a size and shape suitable to receive thrust bearing 105b to provide a support surface for thrust bearing 105b. In conjunction with thrust bearing 105b the shoulders 131 prevent linear translation of inner shaft 109 forward (towards distal end 140d) within outer shaft 102. Downstream of shoulders 131 (near distal end 140d) are shoulders 132. Like shoulders 131, shoulders 132 are configured to receive thrust bearing 105c and provide a support surface for thrust bearing 105c so as to prevent linear translation of shaft 113a and gear 115a backward (towards proximal end 140p) within outer shaft 102. Further downstream of shoulders 132 (at the distal end of outer shaft 102) are surfaces 135. Surfaces 135 are configured to provide a support surface for thrust bearing 105c and prevent linear translation of shaft 113a and gear 115a.

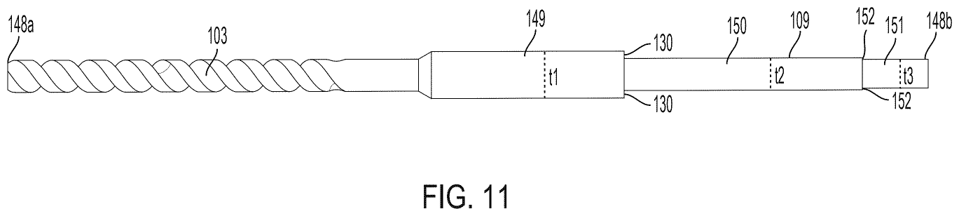

FIG. 11 shows inner shaft 109 removed from surgical device 100. As explained above, inner shaft 109 includes a first end 148a and a second end 148b. Adjacent first end 148a is threaded portion 103. Downstream of threaded portion 103 (near second end 148b) is first segment 149 having a thickness t1. Downstream of first segment 149 is second segment 150 having a thickness t2 and adjoining first segment 149. Thickness t2 is less than thickness t1 forming a shoulder 130 at the juncture where first segment 149 and second segment 150 meet. Shoulder 130 contacts thrust bearing 105b (shown in FIG. 2) and provides a surface against which thrust bearing 105b can rest. Downstream of second segment 150 and shoulder 130 (adjacent second end 148b) is third segment 151. Third segment 151 has a thickness t3, which is less that thickness t1 and thickness t2. At the juncture where second segment 150 and third segment 151 meet is shoulder 152 formed by the difference in thickness t2 and thickness t3. Shoulder 152 provides a surface against which gear shaft 113a can rest.

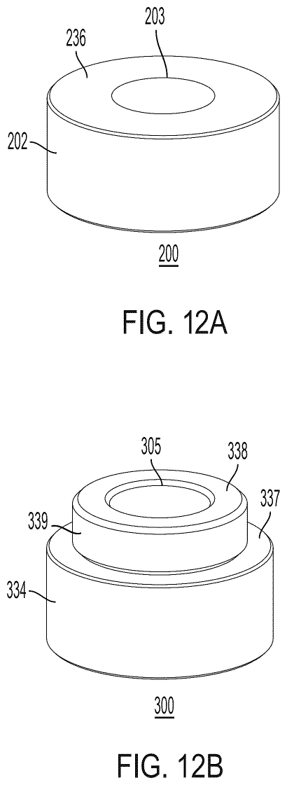

Referring now to FIG. 12A, an embodiment of a thrust bearing 200 is shown. Thrust bearing 200 may function in the same, similar, or substantially similar manner to thrust bearings 105a-b explained above. Thrust bearing 200 includes a cylindrical body 202 and a recess 203 allowing the thrust bearing to radially surround a portion of an inner shaft, such as inner shaft 109. Thrust bearing 200 also includes surfaces 236, which are substantially planar and may abut in a flush manner other components of the surgical device 100 (such as push button 104 or shoulder 131 of outer shaft 102).

Referring now to FIG. 12B, another embodiment of a thrust bearing 300 is shown. Thrust bearing 300 may function in the same, similar, or substantially similar manner to thrust bearings 105c. Thrust bearing 300 includes a cylindrical body 334 and cylindrical body 339. A recess 305 extends through cylindrical body 334 and cylindrical body 339 to allow thrust bearing 300 to radially surround a portion of an inner shaft, such as inner shaft 109. Cylindrical body 339 has a smaller circumference than cylindrical body 334 and is situated on top of surface 337 of cylindrical body 334. Surface 337 may be substantially planar so as to abut in a flush manner with other components of surgical device 100, such as surfaces 135 of outer shaft 102. In addition, cylindrical body 339 has surface 338, which may be substantially planar so as to abut in a flush manner with shoulders 132 of outer shaft 192, for example.

The surfaces 236, 337, and 338 allow the thrust bearings 200 and 300 to act as a bearing surface within outer shaft 102 in order to limit undesirable linear translation of various components within outer shaft 102, as discussed previously herein.

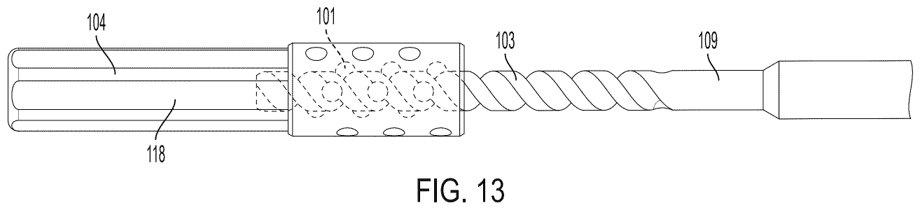

FIG. 13 shows push button 104 operably coupled with inner shaft 109 including one or more driving elements 101 housed in the interior of push button 104 similarly as explained above. In the example illustration, the components within the interior of push button 104 are shown in skeleton lines because ordinarily they would not be visible in this orientation. Other components have been omitted from FIG. 13 for clarity, however, it should be noted that the configuration of push button 104, driving elements 101, and inner shaft 109 discussed in relation to FIG. 13 can apply to the embodiment of surgical device 100 shown in other figures (such as FIGS. 1-12). In the example embodiment, the driving elements 101 are housed in the interior of the push button 104 so that they are in contact with the threads of threaded portion 103 of inner shaft 109. The driving elements 101 may be housed in corresponding indents, pockets or grooves 198 (see FIGS. 3A-C) in the push button 104 so that they are prevented from translating within the push button 104.

In the example embodiment, there are a plurality of driving elements 101 spaced around the threaded portion 103 to evenly distribute forces and facilitate a smooth transfer of the linear motion of push button 104 into rotational motion of threaded shaft 103. As explained previously, the number of driving elements 101 can vary. In some embodiments, there may be a set of three driving elements 101 along a top portion of threaded shaft 103, three driving elements 101 along a front side of threaded shaft 103, three driving elements 101 along a bottom portion of threaded shaft 103, and three driving elements 101 along a back side of threaded shaft 103 (not necessarily visible in FIG. 13) which results in a total of twelve driving elements 101. In this way, the driving elements are sequentially and spatially oriented in a symmetrically distributed way and can facilitate a very smooth type of action which may be important when performing surgery on delicate anatomical features.

As discussed previously in relation to FIGS. 1 and 2, when the push button 104 is actuated the driving elements 101 rotate within their respective indents, pockets, slots, or grooves 198 (see FIGS. 3A-C) causing inner shaft 109 to rotate in a first direction. In addition, when the push button 104 is actuated spring 107 (shown in FIG. 2) is compressed. As inner shaft 109 rotates in the first direction the driving elements 101 move along the threads of the threaded portion 103 in a forward direction towards the distal end 140d (denoted by the arrow d1 in FIG. 2). Conversely, when the push button 104 is released, it is returned to its initial (pre-actuated) position by the return force of compressed spring 107. As the push button 104 returns to its initial position the driving elements 101 rotate in their respective pockets, indents, slots, or grooves 198 (see FIGS. 3A-C) allowing the inner shaft 109 to rotate in a second direction (opposite direction from the first direction) and the driving elements 101 move along the threads of the threaded portion 103 in a backward direction towards the proximal end 140p (denoted by arrow d2 in FIG. 2). In this sense, actuating the push button 104 by moving it linearly forward causes the inner shaft 109 to rotate about axis 143 in a first rotation direction and compress the spring 107 without moving the inner shaft 109 linearly forward. Thereafter, releasing the push button 104 allows the return force of the compressed spring 107 to linearly translate the push button 104 backwards thereby causing the inner shaft 109 to rotate about axis 143 in a second rotation direction opposite the first rotation direction.

The thread pitch of bone piercing tool 119 can be customized for different use cases. For example, in some embodiments the thread pitch of threaded portion 121 of bone piercing tool 119 can be that of a power screw e.g., about 2 mm of linear translation per a single revolution of bone piercing tool 119 about axis 201 to about 12 mm of linear translation per a single revolution of bone piercing tool 119 about axis 201. Thread pitches in this range can be advantageous for use cases where it is desirable to translate the bone piercing tool 119 rapidly i.e., a greater linear distance per revolution of gears 115a-b or, alternatively stated, translate the bone piercing tool 119 a relatively great distance per amount of compression of the push button 104. In other embodiments the thread pitch of threaded portion 121 can be that of a locking screw e.g., 0.35 mm of linear translation per a single revolution of bone piercing tool 119 about axis 201 to about 1.5 mm of linear translation per a single revolution of bone piercing tool 119 about axis 201. In other embodiments, pitches similar to typical machine screws may be employed. Thread pitches in this range can be advantageous for use cases where it is desirable that the bone piercing tool 119 withstand vibration or loosening, such as surgical procedures requiring a high amount of precision. In still further embodiments, thread pitches can be such that threaded portion 121 is configured as a non-locking screw.

As stated in the previous paragraph, the thread pitch of the bone piercing tool 119 can be customized to affect different amounts of linear motion relative to linear motion of the push button 104. For example, in some embodiments, the ratio of bone piercing tool 119 linear translation to push button 104 linear translation can be 1:1 (e.g., 20 mm: 20 mm). In other embodiments the ratio of bone piercing tool 119 linear translation to push button 104 linear translation can be 1:2 (e.g., 10 mm: 20 mm).

The ranges of thread pitches stated above for threaded portion 121 can similarly be applied to threaded portion 103.

It should be understood that various aspects disclosed herein may be combined in different combinations than the combinations specifically presented in the description and accompanying drawings. For example, features, functionality, and components from one embodiment may be combined with another embodiment and vice versa unless the context clearly indicates otherwise. Similarly, features, functionality, and components may be omitted unless the context clearly indicates otherwise. It should also be understood that, depending on the example, certain acts or events of any of the processes or methods described herein may be performed in a different sequence, may be added, merged, or left out altogether (e.g., all described acts or events may not be necessary to carry out the techniques).

Unless otherwise specifically defined herein, all terms are to be given their broadest possible interpretation including meanings implied from the specification as well as meanings understood by those skilled in the art and/or as defined in dictionaries, treatises, etc. It must also be noted that, as used in the specification and the appended claims, the singular forms “a,” “an” and “the” include plural referents unless otherwise specified, and that the terms “comprises” and/or “comprising,” when used in this specification, specify the presence of stated features, elements, and/or components, but do not preclude the presence or addition of one or more other features, steps, operations, elements, components, and/or groups thereof.

Without excluding further possible embodiments, certain example embodiments are summarized in the following clauses:

Clause 1: A surgical device, comprising: an outer shaft extending between a proximal end and a distal end along a central axis; a driving mechanism comprising: an inner shaft having a first threaded portion and extending through the outer shaft along the central axis between the proximal end and the distal end; and one or more driving elements in contact with the first threaded portion of the inner shaft; an angled sleeve positioned on the distal end of the outer shaft and supporting an angled shaft therein that is angled relative to the central axis; a first gear coupled to a distal end of the inner shaft and a second geared meshed with the first gear, the second gear coupled to the angled shaft; a push button coupled to the proximal end of the inner shaft, wherein the push button supports the one or more driving elements and is linearly translatable between a first position and a second position; a bone piercing tool coupled to the angled shaft and rotatable via rotation of the second gear, the bone piercing tool being extendable and retractable along a trajectory between a non-deployed position and a deployed position upon linear translation of the push button, wherein, in the deployed position, a portion of the bone piercing tool extends distally beyond a distal tip of the angled sleeve along the trajectory, wherein, when linearly translating from the first position to the second position, the push button is configured to simultaneously move the one or more driving elements in a forward direction along the first threaded portion so that the inner shaft axially rotates in a first direction and linearly translates the bone piercing tool along the trajectory to the deployed position, wherein, when linearly translating from the second position to the first position, the inner shaft axially rotates in a second direction opposite the first direction and linearly translates the bone piercing tool along the trajectory to the non-deployed position.

Clause 2: The surgical device of clause 1, further comprising a cap coupled to the outer sleeve and having an interior cavity configured to house the push button so that the push button cannot rotate within the cap.

Clause 3: The surgical device of clause 1, wherein the second gear is configured to rotate in a clockwise direction when the inner shaft rotates in the first direction and to rotate in a counterclockwise direction when the inner shaft rotates in the second direction.

Clause 4: The surgical device of clause 1, wherein the bone piercing tool comprises a second threaded portion and the angled sleeve supports one or more posts mated with the second threaded portion and configured to cause the bone piercing tool to linearly move back and forth between the non-deployed position and the deployed position when the second gear rotates.

Clause 5: The surgical device of clause 4, wherein the one or more posts are received in a corresponding pocket of the angled sleeve and the corresponding pockets are each configured to maintain the posts in a corresponding relative position by preventing the posts from linearly translating.

Clause 6: The surgical device of clause 4, wherein the bone piercing tool is configured as an acme screw and wherein the one or more posts are threads.

Clause 7: The surgical device of clause 4, wherein the bone piercing tool is configured as a ball screw and wherein the one or more posts are one or more balls mated with the second threaded portion.

Clause 8: The surgical device of clause 7, wherein the one or more balls comprises exactly two balls and the bone piercing tool is configured as a multi-lead ball screw including exactly one corresponding lead for each ball.

Clause 9: The surgical device of any one of clauses 4-8, wherein the second threaded portion includes a thread diameter such that the second threaded portion forms a non-locking thread.

Clause 10: The surgical device of any one of clauses 1-9, wherein the first threaded portion includes a thread diameter such that the first threaded portion forms a non-locking thread.

Clause 11: The surgical device of any one of clauses 1-10, wherein the push button is configured to be driven between the first position and the second position via a powered device.

Clause 12: The surgical device of any one of clauses 1-11, wherein the first gear and the second gear are oriented relative to each other at an angle in the range of about 15 degrees to about 60 degrees.

Clause 13: The surgical device of any one of clauses 1-12, wherein the inner shaft and the driving elements are configured as a back driving screw.

Clause 14: The surgical device of any one of clauses 1-13, wherein the first threaded portion comprises a threaded profile and the one or more driving elements are teeth that comprise a geometry corresponding to the threaded profile.

Clause 15: The surgical device of any one of clauses 1-14, wherein the bone piercing tool further comprises a trocar tip.

Clause 16: The surgical device of any one of clauses 1-15, further comprising:
a first distal bushing; a second distal bushing; a first cannulated shaft coupled to the first gear; and a second cannulated shaft, coupled to the second gear, wherein the first distal bushing and the second distal bushing are coupled to the second cannulated shaft in such a manner that prevents translational movement of the first and second gears, wherein the first distal bushing is configured as both a thrust bearing and a radial bearing.

Clause 17: The surgical device of any one of clauses 1-16, further comprising one or more bearings configured to prevent the inner shaft from linearly translating along the central axis.

Clause 18: The surgical device of any one of clauses 1-17, further comprising: a compressible helical spring that is compressed when moving the push button from the first position to the second position, wherein, in the second position, the spring is configured to urge the push button towards the first position to linearly translate the push button to the first position, axially rotate the inner shaft in the second direction, and linearly translate the bone piercing tool along the trajectory to the non-deployed position.

Clause 19: A surgical device comprising: a rotational mechanism configured to translate rotational force; and a bone piercing tool coupled to the rotational mechanism and configured to rotate upon translation of rotational force to the bone piercing tool by the rotational mechanism wherein the bone piercing tool includes a threaded portion and one or more posts mated with the threaded portion, the one or more posts being configured to cause the bone piercing tool to linearly move back and forth between a non-deployed position and a deployed position when the rotational mechanism translates rotational force to the bone piercing tool.

Clause 20: A medical device system, comprising: a medical implant comprising at least one fastening hole; and a surgical device comprising: an outer shaft extending between a proximal end and a distal end along a central axis; a driving mechanism comprising: an inner shaft having a first threaded portion and extending through the outer shaft along the central axis between the proximal end and the distal end; and one or more driving elements in contact with the first threaded portion of the inner shaft; an angled sleeve positioned on the distal end of the outer shaft and supporting an angled shaft therein that is angled relative to the central axis; a first gear coupled to a distal end of the inner shaft and a second geared meshed with the first gear, the second gear coupled to the angled shaft; a push button coupled to the proximal end of the inner shaft, wherein the push button supports the one or more driving elements and is linearly translatable between a first position and a second position; a bone piercing tool coupled to the angled shaft and rotatable via rotation of the second gear, the bone piercing tool being extendable and retractable along a trajectory between a non-deployed position and a deployed position upon linear translation of the push button, wherein, in the deployed position, a portion of the bone piercing tool extends distally beyond a distal tip of the angled sleeve along the trajectory, wherein, when linearly translating from the first position to the second position, the push button is configured to simultaneously move the one or more driving elements in a forward direction along the first threaded portion so that the inner shaft axially rotates in a first direction and linearly translates the bone piercing tool along the trajectory to the deployed position, wherein, when linearly translating from the second position to the first position, the inner shaft axially rotates in a second direction opposite the first direction and linearly translates the bone piercing tool along the trajectory to the non-deployed position, and wherein the bone piercing tool is configured to cut holes in bony anatomy corresponding to the size and angularity of the at least one fastening hole.

What is claimed is:

1. A surgical device, comprising:
an outer shaft extending between a proximal end and a distal end along a central axis;
a driving mechanism comprising:
an inner shaft having a first threaded portion and extending through the outer shaft along the central axis between the proximal end and the distal end; and
one or more driving elements in contact with the first threaded portion of the inner shaft;
an angled sleeve positioned on the distal end of the outer shaft and supporting an angled shaft therein that is angled relative to the central axis;
a first gear coupled to a distal end of the inner shaft and a second geared meshed with the first gear, the second gear coupled to the angled shaft;
a push button coupled to the proximal end of the inner shaft, wherein the push button supports the one or more driving elements and is linearly translatable between a first position and a second position;
a bone piercing tool coupled to the angled shaft and rotatable via rotation of the second gear, the bone piercing tool being extendable and retractable along a trajectory between a non-deployed position and a deployed position upon linear translation of the push button,
wherein, in the deployed position, a portion of the bone piercing tool extends distally beyond a distal tip of the angled sleeve along the trajectory,
wherein, when linearly translating from the first position to the second position, the push button is configured to simultaneously move the one or more driving elements in a forward direction along the first threaded portion so that the inner shaft axially rotates in a first direction and linearly translates the bone piercing tool along the trajectory to the deployed position,
wherein, when linearly translating from the second position to the first position, the inner shaft axially rotates in a second direction opposite the first direction and linearly translates the bone piercing tool along the trajectory to the non-deployed position.

2. The surgical device of claim 1, further comprising a cap coupled to the outer sleeve and having an interior cavity configured to house the push button so that the push button cannot rotate within the cap.

3. The surgical device of claim 1, wherein the second gear is configured to rotate in a clockwise direction when the inner shaft rotates in the first direction and to rotate in a counterclockwise direction when the inner shaft rotates in the second direction.

4. The surgical device of claim 1, wherein the bone piercing tool comprises a second threaded portion and the angled sleeve supports one or more posts mated with the second threaded portion and configured to cause the bone piercing tool to linearly move back and forth between the non-deployed position and the deployed position when the second gear rotates.

5. The surgical device of claim 4, wherein the one or more posts are received in a corresponding pocket of the angled sleeve and the corresponding pockets are each configured to maintain the posts in a corresponding relative position by preventing the posts from linearly translating.

6. The surgical device of claim 4, wherein the bone piercing tool is configured as an acme screw and wherein the one or more posts are threads.

7. The surgical device of claim 4, wherein the bone piercing tool is configured as a ball screw and wherein the one or more posts are one or more balls mated with the second threaded portion.

8. The surgical device of claim 7, wherein the one or more balls comprises exactly two balls and the bone piercing tool is configured as a multi-lead ball screw including exactly one corresponding lead for each ball.

9. The surgical device of claim 4, wherein the second threaded portion includes a thread diameter such that the second threaded portion forms a non-locking thread.

10. The surgical device of claim 1, wherein the first threaded portion includes a thread diameter such that the first threaded portion forms a non-locking thread.

11. The surgical device of claim 1, wherein the push button is configured to be driven between the first position and the second position via a powered device.

12. The surgical device of claim 1, wherein the first gear and the second gear are oriented relative to each other at an angle in the range of about 15 degrees to about 60 degrees.

13. The surgical device of claim 1, wherein the inner shaft and the driving elements are configured as a back driving screw.

14. The surgical device of claim 1, wherein the first threaded portion comprises a threaded profile and the one or more driving elements are teeth that comprise a geometry corresponding to the threaded profile.

15. The surgical device of claim 1, wherein the bone piercing tool further comprises a trocar tip.

16. The surgical device of claim 1, further comprising:
a first distal bushing;
a second distal bushing;
a first cannulated shaft coupled to the first gear; and
a second cannulated shaft, coupled to the second gear,
wherein the first distal bushing and the second distal bushing are coupled to the second cannulated shaft in such a manner that prevents translational movement of the first and second gears,
wherein the first distal bushing is configured as both a thrust bearing and a radial bearing.

17. The surgical device of claim 1, further comprising one or more bearings configured to prevent the inner shaft from linearly translating along the central axis.

18. The surgical device of claim 1, further comprising:
a compressible helical spring that is compressed when moving the push button from the first position to the second position,
wherein, in the second position, the spring is configured to urge the push button towards the first position to linearly translate the push button to the first position, axially rotate the inner shaft in the second direction, and linearly translate the bone piercing tool along the trajectory to the non-deployed position.

19. A surgical device comprising:
a rotational mechanism configured to translate rotational force; and
a bone piercing tool coupled to the rotational mechanism and configured to rotate upon translation of rotational force to the bone piercing tool by the rotational mechanism,
wherein the bone piercing tool includes a threaded portion and one or more posts mated with the threaded portion, the one or more posts being configured to cause the bone piercing tool to linearly move back and forth between a non-deployed position and a deployed position when the rotational mechanism translates rotational force to the bone piercing tool.

20. A medical device system, comprising:
a medical implant comprising at least one fastening hole; and
a surgical device comprising:
an outer shaft extending between a proximal end and a distal end along a central axis;
a driving mechanism comprising:
an inner shaft having a first threaded portion and extending through the outer shaft along the central axis between the proximal end and the distal end; and
one or more driving elements in contact with the first threaded portion of the inner shaft;
an angled sleeve positioned on the distal end of the outer shaft and supporting an angled shaft therein that is angled relative to the central axis;
a first gear coupled to a distal end of the inner shaft and a second geared meshed with the first gear, the second gear coupled to the angled shaft;
a push button coupled to the proximal end of the inner shaft, wherein the push button supports the one or more driving elements and is linearly translatable between a first position and a second position;
a bone piercing tool coupled to the angled shaft and rotatable via rotation of the second gear, the bone piercing tool being extendable and retractable along a trajectory between a non-deployed position and a deployed position upon linear translation of the push button,
wherein, in the deployed position, a portion of the bone piercing tool extends distally beyond a distal tip of the angled sleeve along the trajectory,
wherein, when linearly translating from the first position to the second position, the push button is configured to simultaneously move the one or more driving elements in a forward direction along the first threaded portion so that the inner shaft axially rotates in a first direction and linearly translates the bone piercing tool along the trajectory to the deployed position,
wherein, when linearly translating from the second position to the first position, the inner shaft axially rotates in a second direction opposite the first direction and linearly translates the bone piercing tool along the trajectory to the non-deployed position, and wherein the bone piercing tool is configured to cut holes in bony anatomy corresponding to the size and angularity of the at least one fastening hole.
